(19) United States
(12) Patent Application Publication
(10) Pub. No.: US 2005/0031850 A1
(43) Pub. Date: Feb. 10, 2005

(54) Superabsorbent polymer particles

(76) Inventors: Michael Mitchell, Waxhaw, NC (US); Kimberly A. Rausch, Des Plaines, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/493,917

(22) PCT Filed: Nov. 15, 2002
(86) PCT No.: PCT/EP02/12809

Related U.S. Application Data
(60) Provisional application No. 60/332,197, filed on Nov. 21, 2001.

(51) Int. Cl. B32B 5/16; B32B 27/02; A61F 13/15

(52) U.S. Cl. 428/327; 428/402; 604/370; 604/372

(57) ABSTRACT

Superabsorbent polymer particles having a particle size of about 38 to about 300 μm and a median particle size of about 180 μm or less are disclosed. The superabsorbent polymer particles comprise at least one unneutralized acidic water-absorbing resin and at least one unneutralized basic water-absorbing resin. One embodiment is multicomponent superabsorbent particles wherein each particle contains at least one microdomain of the acidic resin in contact with, or in close proximity to, at least one microdomain of the basic resin. Mixed bed of multicomponent superabsorbent gel particles with particles of a second unneutralized water-ab-sorbing resin, and mixed bed of particles of an unneutral-ized acidic water-absorbing resin and an unneutralized basic water-absorbing resin, also are disclosed. Improved diaper cores containing the small particle size superabsorbent polymer particles also are disclosed.

### FIELD OF THE INVENTION

The present invention relates to superabsorbent polymer particles containing at least one unneutralized acidic water-absorbing resin and at least one unneutralized basic water-absorbing resin, and having (a) a particle size of about 38 to about 300 μm and (b) a median particle size of less than about 180 μm. The particles can be multicomponent superabsorbent polymer particles having at least one microdomain of the acidic resin in contact with, or in close proximity to, at least one microdomain of the basic resin. The present invention also relates to mixtures of superabsorbent polymer particles having a small particle, size and (A) containing (i) multicomponent superabsorbent particles and (ii) particles of an unneutralized acidic water-absorbing resin, an unneutralized basic water-absorbing resin, or a mixture thereof, or (B) containing (i) particles of an unneutralized acidic water-absorbing resin and (ii) particles of an unneutralized basic water-absorbing resin.

### BACKGROUND OF THE INVENTION

Water-absorbing resins are widely used in sanitary goods, hygienic goods, wiping cloths, water-retaining agents, dehydrating agents, sludge coagulants, disposable towels and bath mats, disposable door mats, thickening agents, disposable litter mats for pets, condensation-preventing agents, and release control agents for various chemicals. Water-absorbing resins are available in a variety of chemical forms, including substituted and unsubstituted natural and synthetic polymers, such as hydrolysis products of starch acrylonitrile graft polymers, carboxymethylcellulose, crosslinked polyacrylates, sulfonated polystyrenes, hydrolyzed polyacrylamides, polyvinyl alcohols, polyethylene oxides, polyvinylpyrrolidones, and polyacrylonitriles.

Such water-absorbing resins are termed “superabsorbent polymers,” or SAPs, and typically are lightly crosslinked hydrophilic polymers. SAPs generally are discussed in Goldman et al. U.S. Pat. Nos. 5,669,894 and 5,559,335, each incorporated herein by reference. SAPs can differ in their chemical identity, but all SAPs are capable of absorbing and retaining amounts of aqueous fluids equivalent to many times their own weight, even under moderate pressure. For example, SAPs can absorb one hundred times their own weight, or more, of distilled water. The ability to absorb aqueous fluids under a confining pressure is an important requirement for an SAP used in a hygienic article, such as a diaper.

As used here and hereafter, the term “SAP particles” refers to superabsorbent polymer particles in the dry state, i.e., particles containing from no water up to an amount of water less than the weight of the particles. The terms “SAP gel” or “SAP hydrogel” refer to a superabsorbent polymer in the hydrated state, i.e., particles that have absorbed at least their weight in water, and typically several times their weight in water.

The development of highly absorbent, SAP-containing articles for use as disposable diapers, adult incontinence pads and briefs, and catamenial products, such as sanitary napkins, is the subject of substantial commercial interest. A highly desired characteristic of such absorbent articles is thinness. For example, thinner diapers are less bulky to wear, fit better under clothing, and are less noticeable. Article packaging also is more compact, which makes the diapers easier for the consumer to carry and store. Packaging compactness also results in reduced distribution costs for the manufacturer and distributor, including less required shelf space per diaper unit.

A variety of parameters effect the ability of an SAP particle to rapidly absorb a large amount of a fluid, and then to retain the absorbed fluid under various stresses. Optimization of these parameters allows a reduction in amounts of cellulosic fiber present in a diaper core, which in turn reduces the overall bulk of the diaper. SAP particles, therefore, are designed in an attempt to optimize absorption capacity, absorption rate, acquisition time, gel strength, and permeability.

The present invention is directed to the surprising and unexpected finding that smaller SAP particle size distribution improves absorption and retention properties, and reduces or eliminates the amount of cellulosic fibers, or fluff, in a diaper core. Rapid diffusive absorption of a fluid by an SAP requires small particle radius, but rapid convective flow requires large pores that result from packing together large particles. This conflict in properties can be overcome by a proper selection of the SAP particles and the SAP particle size distribution.

Absorbent articles contain a relatively low amount (e.g., less than about 50% by weight) of SAP particles for several reasons. First, SAPs employed in present absorbent articles lack an absorption rate that allows the SAP particles to quickly absorb body fluids, especially in “gush” situations. This necessitates the inclusion of fibers, typically wood pulp fibers, in the absorptive core of the article as temporary reservoirs to hold the discharged fluids until absorbed by the hydrogel-forming absorbent polymer.

In order to manufacture diaper cores substantially, or completely, free of cellulosic fiber, a continuous zone of SAP particles is required. However, because of the nature of SAP particles, it is impossible to combine features like high absorption capacity and high gel strength within one SAP product because improving one feature adversely affects the other. For example, in order to provide a high absorption capacity, the degree of SAP crosslinking has to be sufficiently low to enable long flexible polymer chains to absorb large quantities of fluids. But, the degree of crosslinking also determines the gel strength of the superabsorbent polymer. In personal care products, SAP hydrogels of a relatively high gel strength are needed because of the mechanical forces applied by individuals wearing personal care products. High gel strength is obtained by higher degrees of crosslinking, and for that reason, a well-defined lower crosslinking limit exists to produce useful superabsorbents.

More importantly, many SAPs exhibit gel blocking. “Gel blocking” occurs when the SAP particles are wetted, and the SAP particles swell to inhibit fluid transmission to other regions of the absorbent structure. Wetting of these other regions of the absorbent member takes place via a slow diffusion process. Gel blocking can be a particularly acute problem if the SAP particles do not have adequate gel strength and deform or spread under stress once the particles swell with absorbed fluid. In practical terms, the acquisition of fluids by the absorbent article is much slower than the rate at which a fluid is discharged, especially in a gush situation. Leakage from the absorbent article can take place well before the SAP particles in the absorbent article are fully saturated or before the fluid can diffuse or wick past the “blocking” particles into the remainder of the absorbent core.

The gel blocking phenomena necessitates the use of a fibrous matrix in which the SAP particles are dispersed. The fibrous matrix separates the SAP particles from one another. The fibrous matrix also provides a capillary structure that allows fluid to reach SAP located in regions of the core remote from the initial fluid discharge point. However, dispersing a relatively low amount of the SAP in a fibrous matrix to minimize or avoid gel blocking reduces the overall fluid storage capacity of absorbent cores. Overall, using lower amounts of an SAP limits the advantage of the SAP, i.e., an ability to absorb and retain large quantities of body fluids per given volume.

In addition to increasing gel strength, other physical and chemical SAP properties have been manipulated to decrease gel blocking. One property is particle size, and especially particle size distribution, of the SAP used in the fibrous matrix. Generally, the transport of a fluid away from the area of initial discharge is improved as the median particle size of the SAP particles increases. However, as the particle size distribution increases, a decay in fluid acquisition time is observed because of a reduction of the surface area of the SAP particles.

For example, an SAP having a particle size distribution such that the SAP particles have a mass median particle size equal to or greater than about 400 microns have been mixed with hydrophilic fibrous materials. This admixture minimizes gel blocking and helps maintain an open capillary structure within the absorbent structure to enhance planar transport of fluids away from the area of initial discharge to the remainder of the absorbent core (see WO 98/37149). In addition, the particle size distribution of the SAP can be controlled to improve absorbent capacity and efficiency of the particles employed in the absorbent structure (see U.S. Pat. Nos. 5,047,023 and 5,061,259). However, U.S. Pat. No. 5,047,023 discloses that adjusting the particle size distribution does not, by itself, provide absorbent articles containing a relatively high amount of SAP particles.

For absorbent cores containing a relatively high amount of SAP particles, other SAP properties also are important. It has been found that the openness, or porosity, of the hydrogel layer formed when the SAP swells in the presence of body fluids helps determine the ability of an SAP to acquire and transport a fluid, especially when the SAP is present in high amounts in the absorbent core. Porosity refers to the fractional volume of a particle that is not occupied by solid material. For a hydrogel layer formed entirely from an SAP, porosity is the fractional volume of the layer that is not occupied by hydrogel. For an absorbent structure containing the hydrogel, as well as other components, porosity is the fractional volume (also referred to as void volume) that is not occupied by the hydrogel or other solid components (e.g., cellulosic fibers).

The most commonly used SAP for absorbing electrolyte-containing liquids, such as urine, is neutralized polyacrylic acid, i.e., containing at least 50%, and up to 100%, neutralized carboxyl groups. Neutralized polyacrylic acid, however, is susceptible to salt poisoning. Therefore, to provide an SAP that is less susceptible to salt poisoning, an SAP different from neutralized polyacrylic acid must be used.

The salt poisoning effect has been explained as follows. Water-absorption and water-retention characteristics of SAPs are attributed to the presence of ionizable functional groups in the polymer structure. The ionizable groups typically are carboxyl groups, a high proportion of which are in the salt form when the polymer is dry, and which undergo dissociation and solvation upon contact with water. In the dissociated state, the polymer chain contains a plurality of functional groups having the same electric charge and, thus, repel one another. This electronic repulsion leads to expansion of the polymer structure, which, in turn, permits further absorption of water molecules. Polymer expansion, however, is limited by the crosslinks in the polymer structure, which are present in a sufficient number to prevent solubilization of the polymer.

It is theorized that the presence of a significant concentration of electrolytes interferes with dissociation of the ionizable functional groups, and leads to the “salt poisoning” effect. Dissolved ions, such as sodium and chloride ions, therefore, have two effects on SAP gels. The ions screen the polymer charges and the ions eliminate the osmotic imbalance due to the presence of counter ions inside and outside of the gel. The dissolved ions, therefore, effectively convert an ionic gel into a nonionic gel, and swelling properties are lost.

Investigators have attempted to counteract the salt poisoning effect and thereby improve the performance of SAPs with respect to absorbing electrolyte-containing liquids, such as menses and urine. For example, Tanaka et al. U.S. Pat. No. 5,274,018 discloses an SAP composition comprising a swellable hydrophilic polymer, such as polyacrylic acid, and an amount of an ionizable surfactant sufficient to form at least a monolayer of surfactant on the polymer. In another embodiment, a cationic gel, such as a gel containing quaternized ammonium groups and in the hydroxide (i.e., OH) form, is admixed with an anionic gel (i.e., a polyacrylic acid) to remove electrolytes from the solution by ion exchange. Quaternized ammonium groups in the hydroxide form are very difficult and time-consuming to manufacture, thereby limiting the practical use of such cationic gels.

Wong U.S. Pat. No. 4,818,598 discloses the addition of a fibrous anion exchange material, such as DEAE (diethylaminoethyl) cellulose, to a hydrogel, such as a polyacrylate, to improve absorption properties. The ion exchange resin “pretreats” the saline solution (e.g., urine) as the solution flows through an absorbent structure (e.g., a diaper). This pretreatment removes a portion of the salt from the saline. The conventional SAP present in the absorbent structure then absorbs the treated saline more efficiently than untreated saline. The ion exchange resin, per se, does not absorb the saline solution, but merely helps overcome the “salt poisoning” effect.

WO 96/17681 discloses admixing discrete anionic SAP particles, such as polyacrylic acid, with discrete polysaccharide-based cationic SAP particles to overcome the salt poisoning effect. Similarly, WO 96/15163 discloses combining a cationic SAP having at least 20% of the functional groups in a basic (i.e., OH) form with a cationic exchange resin, i.e., a nonswelling ion exchange resin, having at least 50% of the functional groups in the acid form. WO 96/15180 discloses an absorbent material comprising an anionic SAP, e.g., a polyacrylic acid, and an anion exchange resin, i.e., a nonswelling ion exchange resin. Such admixtures of resins have been referred to as “mixed bed” systems. Also see WO 96/15162 and WO 98/37149.

It would be desirable to provide SAP particles that exhibit exceptional water absorption and retention properties, especially with respect to electrolyte-containing liquids, and thereby overcome the salt poisoning effect. In addition, it would be desirable to provide SAP particles that have an ability to absorb liquids quickly, demonstrate good fluid permeability and conductivity into and through an SAP particle and an absorbent core containing SAP particles, and have a high gel strength, such that the hydrogel formed from the SAP particles does not deform or flow under an applied stress or pressure.

### SUMMARY OF THE INVENTION

The present invention is directed to SAP particles comprising at least one unneutralized acidic water-absorbing resin, such as a polyacrylic acid, and at least one unneutral-ized basic water-absorbing resin, such as a poly(vinylamine) or a polyethyleneimine, and (a) having a particle size of about 38 to about 300 μm and (b) a median particle size of less than about 180 μm. The SAP particles can be (a) multicomponent superabsorbent particles disclosed in U.S. Pat. Nos. 6,072,101; 6,159,591; 6,222,091; and 6,235,965, each incorporated herein by reference, (b) a mixture of (i) multicomponent superabsorbent particles and (ii) particles of an unneutralized acidic water-absorbing resin, unneutralized basic water-absorbing resin, or a mixture thereof, and (c) a mixture of (i) particles of an unneutralized acid water-absorbing resin and (ii) particles of an unneutral-ized basic water-absorbing resin.

More particularly, in one embodiment, the present invention is directed to multicomponent SAP particles containing at least one discrete microdomain of at least one acidic water-absorbing resin in contact with, or in close proximity to, at least one microdomain of at least one basic water-absorbing resin, and (a) having a particle size of about 38 to about 300 μm and (b) a median particle size of less than about 180 μm. The multicomponent SAP particles can contain a plurality of microdomains of the acidic water-absorbing resin and/or the basic water-absorbing resin dispersed throughout the particle. The acidic resin can be a strong or a weak acidic resin. Similarly, the basic resin can be a strong or a weak basic resin. A preferred SAP contains one or more microdomains of at least one weak acidic resin and one or more microdomains of at least one weak basic resin.

Accordingly, one aspect of the present invention is to provide SAP particles having a small, defined particle size, and that have a high absorption rate, have good permeability and gel strength, overcome the salt poisoning effect, and demonstrate an improved ability to absorb and retain electrolyte-containing liquids, such as saline, blood, urine, and menses. The multicomponent SAP particles contain discrete microdomains of acidic and basic resins, and during hydration, the particles resist coalescence and remain fluid permeable.

Yet another aspect of the present invention is to provide an SAP material A comprising a mixture containing (i) multicomponent SAP particles, and (ii) particles of a second water-absorbing resin selected from the group consisting of an unneutralized acidic water-absorbing resin, an unneutralized basic water-absorbing resin, and a mixture thereof, and (a) having a particle size of about 38 to about 300 μm and (b) a median particle size of less than about 180 m. The mixture contains about 10% to about 90%, by weight, multicomponent SAP particles and about 10% to about 90%, by weight, particles of the second water-absorbing resin.

Another aspect of the present invention is to provide an SAP material B comprising a mixture containing (i) particles of an unneutralized acidic water-absorbing resin and (ii) particles of an unneutralized basic water-absorbing resin, and (a) having a particle size of about 38 to about 300 μm and (b) a median particle size of less than about 180 μm. The mixture contains about 10% to about 90%, by weight, acidic resin particles and about 10% to about 90%, by weight, basic resin particles.

Still another aspect of the present invention is to provide absorbent articles, such as diapers and catamenial devices, having an absorbent core comprising multicomponent SAP particles or an SAP material A or B of the present invention and having the recited particle size range and median. The absorbent article comprises a core, wherein the core contains greater than 50%, and up to 100%, by weight, of the multicomponent SAP particles or SAP material A or B.

These and other aspects and advantages of the present invention will become apparent from the following detailed description of the preferred embodiments.

### BRIEF DESCRIPTION OF THE DRAWINGS

FIG. 1 is a schematic diagram of a water-absorbing particle containing microdomains of a first resin dispersed in a continuous phase of a second resin;

FIG. 2 is a schematic diagram of a water-absorbing particle containing microdomains of a first resin and microdomains of a second resin dispersed throughout the particle;

FIG. 3 is a cross section of an absorbent article having a core containing 100% by weight SAP particles;

FIG. 4 is a graph of rewet (grams) vs. median particle size (μm) for a third and fourth insult test on cores containing 60% (by weight) LAF and 40% cellulosic fluff;

FIG. 5 is a graph of acquisition time (seconds) vs. median particle size (μm) for a second, third, and fourth insult test on cores containing 60% (by weight) LAF and 40% cellulosic fluff;

FIG. 6 is a graph of permeability (SFC) and free swell rate (FSR) vs. particle size (μm) for multicomponent SAP particles containing 55 wt % PAA (DN=0) and 45 wt % PVAm (DN=0);

FIGS. 7 and 8 contain bar graphs of rewet (grams) and acquisition rate (ml/sec), respectively, vs. first through third insult tests on fluffless diaper cores containing LAF, with and without an acquisition layer, and on a comparative diaper core; and

FIGS. 9 and 10 are graphs of rewet (grams) and acquisition rate (ml/sec), respectively, vs. median particle size for diaper cores containing SAF and an acquisition layer, and for a comparative diaper core.

### DETAILED DESCRIPTION OF THE PREFERRED EMBODIMENTS

The present invention is directed to SAP particles containing an unneutralized acidic water-absorbing resin and an unneutralized basic water-absorbing resin. As used herein, the term “unneutralized” is defined as a water-absorbing resin neutralized 0% to 50%. The SAP particles have a small particle size of about 30 to about 300 μm, and a median particle size of less than about 180 μm.

In one embodiment, the present invention is directed to multicomponent SAP particles containing at least one microdomain of an acidic water-absorbing resin in close proximity to, and preferably in contact with, at least one microdomain of a basic water-absorbing resin. Each particle contains one or more microdomains of an acidic resin and one or more microdomains of a basic resin. The microdomains can be distributed nonhomogeneously or homogeneously throughout each particle. The multicomponent SAP particles of the present invention have a particle size of about 38 to about 300 μm and have a median particle size less than about 180 μm.

Each multicomponent SAP particle contains at least one acidic water-absorbing resin and at least one basic water-absorbing resin. In one embodiment, the SAP particles consist essentially of acidic resins and basic resins, and contain microdomains of the acidic and/or basic resins. In another embodiment, microdomains of the acidic and basic resins are dispersed in an absorbent matrix resin.

The multicomponent SAP particles of the present invention are not limited to a particular structure or shape. However, it is important that substantially each multicomponent SAP particle contains at least one microdomain of an acidic water-absorbing resin and at least one microdomain of a basic water-absorbing resin in close proximity to one another. Improved water absorption and retention, and improved fluid permeability through and between multicomponent SAP particles, are observed as long as the acidic resin microdomain and the basic resin microdomain are in close proximity within the particle. In a preferred embodiment, the microdomains of acidic and basic resin are in contact.

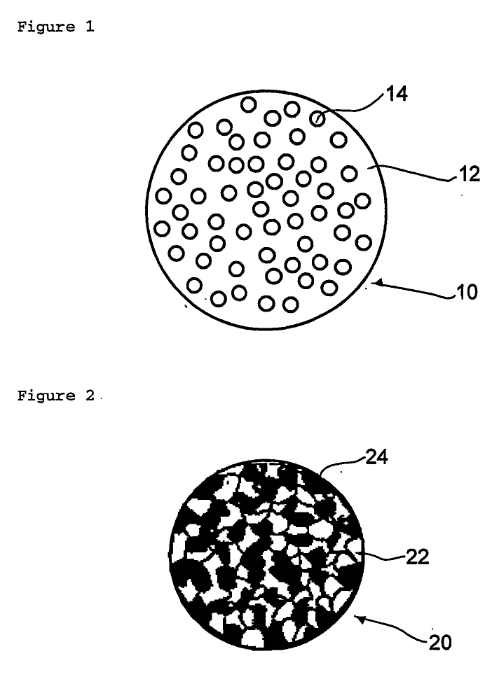

In one embodiment, the multicomponent SAP particles of the present invention can be envisioned as one or more microdomains of an acidic resin dispersed in a continuous phase of a basic resin, or as one or more microdomains of a basic resin dispersed in a continuous acid resin. These idealized multicomponent SAP particles are illustrated in FIG. 1 showing an SAP particle 10 having discrete microdomains 14 of a dispersed resin in a continuous phase of a second resin 12. If microdomains 14 comprise an acidic resin, then continuous phase 12 comprises a basic resin. Conversely, if microdomains 14 comprise a basic resin, then continuous phase 12 is an acidic resin.

In another embodiment, the SAP particles are envisioned as microdomains of an acidic resin and microdomains of a basic resin dispersed throughout each particle, without a continuous phase. This embodiment is illustrated in FIG. 2, showing an idealized multicomponent SAP particle 20 having a plurality of microdomains of an acidic resin 22 and a plurality of microdomains of a basic resin 24 dispersed throughout particle 20.

In yet another embodiment, microdomains of the acidic and basic resins are dispersed throughout a continuous phase comprising a matrix resin. This embodiment also is illustrated in FIG. 1 wherein multicomponent SAP particle 10 contains one or more microdomains 14, each an acidic resin or a basic resin, dispersed in a continuous phase 12 of a matrix resin. Additional embodiments of multicomponent SAP particles are disclosed in U.S. Pat. Nos. 6,072,101; 6,159,591; 6,235,965; and 6,222,091, each incorporated herein by reference.

The multicomponent SAP particles of the present invention comprise an acidic resin and a basic resin in a eight ratio of about 90:10 to about 10:90, and preferably about 20:80 to about 80:20. To achieve the full advantage of the present invention, the weight ratio of acidic resin to basic resin in a multicomponent SAP particle is about 30:70 to about 70:30. The acidic and basic resins can be distributed homogeneously or nonhomogeneously throughout the SAP particle.

The present multicomponent SAP particles contain at least about 50%, and preferably at least about 70%, by weight of acidic resin plus basic resin. To achieve the full advantage of the present invention, a multicomponent SAP particle contains about 80% to 100% by weight of the acidic resin plus basic resin. Components of the present SAP particles, other than the acidic and basic resin, typically, are matrix resins or other minor optional ingredients.

The multicomponent SAP particles, and the particles of SAP materials A and B, can be in any form, either regular or irregular, such as granules, fibers, beads, powders, or flakes, or any other desired shape. In embodiments wherein the multicomponent SAP is prepared using an extrusion step, the shape of the SAP is determined by the shape of the extrusion die. The shape of the SAP particles also can be determined by other physical operations, such as milling or by the method of preparing the particles, such as agglomeration.

In accordance with an important feature of the present invention, the SAP particles utilized in the present invention have a particle size of about 38 to about 300 microns (μm), and preferably about 75 to about 275 μm. To achieve the full advantage of the present invention, the SAP particles have a particle size of about 100 to about 250 μm. The SAP particles also have a median particle size of less than about 180 μm, and preferably less than about 150 μm. To achieve the full advantage of the present invention, the SAP particles have a median particle size of less than about 125 μm. In preferred embodiments, the SAP particles are in the form of a granule or a bead.

For the SAP particles described above, particle size is defined as the dimension determined by sieve size analysis. Thus, for example, a particle that is retained on a U.S.A. Standard Testing Sieve with 250 micron openings (e.g., No. 60 U.S. Series Alternate Sieve Designation) is considered to have a size greater than 250 microns; a particle that passes through a sieve with 250 micron openings and is retained on a sieve with 125 micron openings (e.g., No. 120 U.S. Series Alternate Sieve Designation) is considered to have a particle size between 125 and 250 microns; and a particle that passes through a sieve with 125 micron openings is considered to have a size less than 125 microns.

The median particle size of a given sample of SAP is defined as the particle size that divides the sample in half on a mass basis, i.e., one-half of the sample has a particle size greater than the mass median size. A standard particle-size plotting method (wherein the cumulative weight percent of the particle sample retained on or passed through a given sieve size opening is plotted versus sieve size opening on probability paper) typically is used to determine median particle size when the 50% mass value does not correspond to the size opening of a U.S.A. Standard Testing Sieve. Methods for determining the particle size of the SAP particles are further described in U.S. Pat. No. 5,061,259, incorporated by reference.

A microdomain is defined as a volume of an acidic resin or a basic resin that is present in a multicomponent SAP particle. Because each multicomponent SAP particle contains at least one microdomain of an acidic resin, and at least one microdomain of a basic resin, a microdomain has a volume that is less than the volume of the multicomponent SAP particle. A microdomain, therefore, can be as large as about 90% of the volume of a multicomponent SAP particle.

Typically, a microdomain has a diameter of about 100 μm or less. To achieve the full advantage of the present invention, a microdomain has a diameter of about 20 μm or less. The multicomponent SAP particles also contain microdomains that have submicron diameters, e.g., microdomain diameters of less than 1 μm, preferably less than 0.1 μm, to about 0.01 μm.

In another embodiment, the multicomponent SAP particles are in the shape of a fiber, i.e., an elongated, acicular SAP particle. The fiber is in the shape of a cylinder, for example, having a minor dimension (i.e., diameter) and a major dimension (i.e., length). Cylindrical multicomponent SAP fibers have a minor dimension (i.e., diameter of the fiber) less than about 250 μm, and down to about 38 μm. The cylindrical SAP fibers have a relatively short major dimension, for example, about 100 to about 300 μm.

A multicomponent SAP particle can be in a form wherein a microdomain of an acidic water-absorbing resin is in contact with a microdomain of a basic water-absorbing resin. In another embodiment, the SAP multicomponent particle can be in a form wherein at least one microdomain of an acidic water-absorbing resin is dispersed in a continuous phase of a basic water-absorbing resin. Alternatively, the multicomponent SAP can be in a form wherein at least one microdomain of a basic resin is dispersed in a continuous phase of an acidic resin. In another embodiment, at least one microdomain of one or more acidic resin and at least one microdomain of one or more basic resin comprise the entire SAP particle, and neither type of resin is considered the dispersed or the continuous phase. In yet another embodiment, at least one microdomain of an acidic resin and at least one microdomain of a basic resin are dispersed in a matrix resin.

An acidic water-absorbing resin present in a multicomponent SAP particle can be either a strong or a weak acidic water-absorbing resin. The acidic water-absorbing resin can be a single resin, or a mixture of resins. The acidic resin can be a homopolymer or a copolymer. The identity of the acidic water-absorbing resin is not limited as long as the resin is capable of swelling and absorbing at least ten times its weight in water, when in a neutralized form. The acidic resin is present in its acidic, or unneutral-ized, form, i.e., about 50% to 100% of the acidic moieties are present in the free acid form. As illustrated hereafter, although the free acid form of a acidic water-absorbing resin is generally a poor water absorbent, the combination of an acidic resin and a basic resin either in a multicomponent SAP particle or a mixed bed system provides excellent water absorption and retention properties.

The acidic water-absorbing resin typically is a lightly crosslinked acrylic-type resin, such as lightly crosslinked polyacrylic acid. The lightly crosslinked acidic resin conventionally is prepared by polymerizing an acidic monomer containing an acyl moiety, e.g., acrylic acid, or a moiety capable of providing an acid group, i.e., acrylonitrile, in the presence of a crosslinker, i.e., a polyfunctional organic compound. The acidic resin can contain other copolymerizable units, i.e., other monoethylenically unsaturated comonomers, well known in the art, as long as the polymer is substantially, i.e., at least 10%, and preferably at least 25%, acidic monomer units. To achieve the full advantage of the present invention, the acidic resin contains at least 50%, and more preferably, at least 75%, and up to 100%, acidic monomer units. The other copolymerizable units can, for example, help improve the hydrophilicity of the polymer.

Ethylenically unsaturated carboxylic acid and carboxylic acid anhydride monomers useful in the acidic water-absorbing resin include acrylic acid, methacrylic acid, ethacrylic acid, á-chloroacrylic acid, á-cyanoacrylic acid, â-methylacrylic acid (crotonic acid), á-phenylacrylic acid, â-acryloxypropionic acid, sorbic acid, á-chlorosorbic acid, angelic acid, cinnamic acid, p-chlorocinnamic acid, â-stearylacrylic acid, itaconic acid, citraconic acid, mesaconic acid, glutaconic acid, aconitic acid, maleic acid, furmaric acid, tricarboxyethylene, and maleic anhydride.

Ethylenically unsaturated sulfonic acid monomers include aliphatic or aromatic vinyl sulfonic acids, such as vinylsulfonic acid, allyl sulfonic acid, vinyl toluene sulfonic acid, styrene sulfonic acid, acrylic and methacrylic sulfonic acids, such as sulfoethyl acrylate, sulfoethyl methacrylate, sulfopropyl acrylate, sulfopropyl methacrylate, 2-hydroxy-3-methacryloxypropyl sulfonic acid, and 2-acrylamide-2-methylpropane sulfonic acid.

As set forth above, polymerization of acidic monomers, and copolymerizable monomers, if present, most commonly is performed by free radical processes in the presence of a polyfunctional organic compound. The acidic resins are crosslinked to a sufficient extent such that the polymer is water insoluble. Crosslinking renders the acidic resins substantially water insoluble, and, in part, serves to determine the absorption capacity of the resins. For use in absorption applications, an acidic resin is lightly crosslinked, i.e., has a crosslinking density of less than about 20%, preferably less than about 10%, and most preferably about 0.01% to about 7%.

A crosslinking agent most preferably is used in an amount of less than about 7 wt %, and typically about 0.1 wt % to about 5 wt %, based on the total weight of monomers. Examples of crosslinking polyvinyl monomers include, but are not limited to, polyacrylic (or polymethacrylic) acid esters represented by the following formula (III); and bisacrylamides, represented by the following formula (IV). 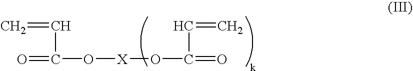
wherein x is ethylene, propylene, trimethylene, cyclohexyl, hexamethylene, 2-hydroxypropylene, —(CH₂CH₂O)ₙCH₂CH₂₋, or 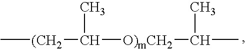
n and m are each an integer 5 to 40, and k is 1 or 2; 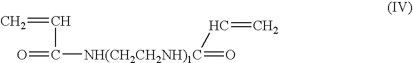
wherein 1 is 2 or 3.

The compounds of formula (III) are prepared by reacting polyols, such as ethylene glycol, propylene glycol, trimethylolpropane, 1,6-hexanediol, glycerin, pentaerythritol, polyethylene glycol, or polypropylene glycol, with acrylic acid or methacrylic acid. The compounds of formula (IV) are obtained by reacting polyalkylene polyamines, such as diethylenetriamine and triethylenetetramine, with acrylic acid.

Specific crosslinking monomers include, but are not limited to, 1,4-butanediol diacrylate, 1,4-butanediol dimethacrylate, 1,3-butylene glycol diacrylate, 1,3-butylene glycol dimethacrylate, diethylene glycol diacrylate, diethylene glycol dimethacrylate, ethoxylated bisphenol A diacrylate, ethoxylated bisphenol A dimethacrylate, ethylene glycol dimethacrylate, 1,6-hexanediol diacrylate, 1,6-hexanediol dimethacrylate, neopentyl glycol dimethacrylate, polyethylene glycol diacrylate, polyethylene glycol dimethacrylate, triethylene glycol diacrylate, triethylene glycol dimethacrylate, tripropylene glycol diacrylate, tetraethylene glycol diacrylate, tetraethylene glycol dimethacrylate, dipentaerythritol pentaacrylate, pentaerythritol tetraacrylate, pentaerythritol triacrylate, trimethylolpropane triacrylate, trimethylolpropane trimethacrylate, tris(2-hydroxyethyl)isocyanurate triacrylate, tris(2-hydroxyethyl)isocyanurate trimethacrylate, divinyl esters of a polycarboxylic acid, diallyl esters of a polycarboxylic acid, triallyl terephthalate, diallyl maleate, diallyl fumarate, hexamethylenebismaleimide, trivinyl trimellitate, divinyl adipate, diallyl succinate, a divinyl ether of ethylene glycol, cyclopentadiene diacrylate, tetraallyl ammonium halides, or mixtures thereof. Compounds such as divinylbenzene and divinyl ether also can be used to crosslink the poly(dialkylaminoalkyl acrylamides). Especially preferred crosslinking agents are N,N′-methylenebisacrylamide, N,N′-methylenebismethacrylamide, ethylene glycol dimethacrylate, and trimethylolpropane triacrylate.

The acidic resin, either strongly acidic or weakly acidic, can be any resin that acts as an SAP in its neutralized form. The acidic resins typically contain a plurality of carboxylic acid, sulfonic acid, phosphonic acid, phosphoric acid, and/or sulfuric acid moieties. Examples of acidic resins include, but are not limited to, polyacrylic acid, hydrolyzed starch-acrylonitrile graft copolymers, starch-acrylic acid graft copolymers, saponified vinyl acetate-acrylic ester copolymers, hydrolyzed acrylonitrile copolymers, hydrolyzed acrylamide copolymers, ethylene-maleic anhydride copolymers, isobutylene-maleic anhydride copolymers, poly(vinylsulfonic acid), poly(vinylphosphonic acid), poly(vinylphosphoric acid), poly(vinylsulfuric acid), sulfonated polystyrene, poly(aspartic acid), poly(lactic acid), and mixtures thereof. The preferred acidic resins are the polyacrylic acids.

The multicomponent SAPs can contain individual microdomains that: (a) contain a single acidic resin or (b) contain more than one, i.e., a mixture, of acidic resins. The multicomponent SAPs also can contain microdomains wherein, for the acidic component, a portion of the acidic microdomains comprise a first acidic resin or acidic resin mixture, and the remaining portion comprises a second acidic resin or acidic resin mixture.

Analogous to the acidic resin, the basic water-absorbing resin in the present SAP particles can be a strong or weak basic water-absorbing resins. The basic water-absorbing resin can be a single resin or a mixture of resins. The basic resin can be a homopolymer or a copolymer. The identity of the basic resin is not limited as long as the basic resin is capable of swelling and absorbing at least 10 times its weight in water, when in a charged form. The weak basic resin typically is present in its free base, or unneutral-ized, form, i.e., about 50% to about 100% of the basic moieties, e.g., amino groups, are present in a neutral, uncharged form. The strong basic resins typically are present in the hydroxide (OH) or bicarbonate (HCO₃) form.

The basic water-absorbing resin typically is a lightly crosslinked acrylic type resin, such as a poly(vinylamine) or a poly(dialkylaminoalkyl (meth)acrylamide). The basic resin also can be a polymer such as a lightly crosslinked polyethylenimine, a poly(allylamine), a poly(allylguanidine), a poly(dimethyldiallylammonium hydroxide), a quaternized polystyrene derivative, such as 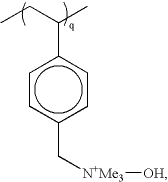
a guanidine-modified polystyrene, such as 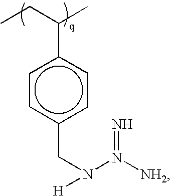
a quaternized poly((meth)acrylamide) or ester analog, such as 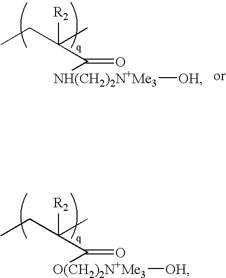
wherein Me is methyl, R₂ is hydrogen or methyl, n is a number 1 to 8, and q is a number from 10 to about 100,000, or a poly(vinylguanidine), i.e., poly(VG), a strong basic water-absorbing resin having the general structural formula (V) 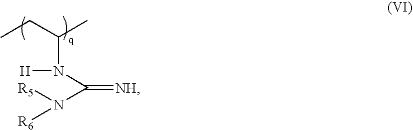
wherein q is a number from 10 to about 100,000, and R₅ and R₆, independently, are selected from the group consisting of hydrogen, C₁-C₄ alkyl, C₃-C₆ cycloalkyl, benzyl, phenyl, alkyl-substituted phenyl, naphthyl, and similar aliphatic and aromatic groups. The lightly crosslinked basic water-absorbing resin can contain other copolymerizable units and is crosslinked using a polyfunctional organic compound, as set forth above with respect to the acidic water-absorbing resin.

A basic water-absorbing resin used in the present SAP particles typically contains an amino or a guanidino group. Accordingly, a water-soluble basic resin also can be crosslinked in solution by suspending or dissolving an uncrosslinked basic resin in an aqueous or alcoholic medium, then adding a di- or polyfunctional compound capable of crosslinking the basic resin by reaction with the amino groups of the basic resin. Such crosslinking agents include, for example, multifunctional aldehydes (e.g., glutaraldehyde), multifunctional acrylates (e.g., butanediol diacrylate, TMPTA), halohydrins (e.g., epichlorohydrin), dihalides (e.g., dibromopropane), disulfonate esters (e.g., ZA(O₂)O—(CH₂)ₙ—OS(O)₂Z, wherein n is 1 to 10, and Z is methyl or tosyl), multifunctional epoxies (e.g., ethylene glycol diglycidyl ether), multifunctional esters (e.g., dimethyl adipate), multifunctional acid halides (e.g., oxalyl chloride), multifunctional carboxylic acids (e.g., succinic acid), carboxylic acid anhydrides (e.g., succinic anhydride), organic titanates (e.g., TYZOR AA from DuPont), melamine resins (e.g., CYMEL 301, CYMEL 303, CYMEL 370, and CYMEL 373 from Cytec Industries, Wayne, N.J.), hydroxymethyl ureas (e.g., N,N′-dihydroxymethyl-4,5-dihydroxyethyleneurea), and multifunctional isocyanates (e.g., toluene diisocyanate or methylene diisocyanate). Crosslinking agents also are disclosed in Pinschmidt, Jr. et al. U.S. Pat. No. 5,085,787, incorporated herein by reference, and in EP 450 923.

Conventionally, the crosslinking agent is water or alcohol soluble, and possesses sufficient reactivity with the basic resin such that crosslinking occurs in a controlled fashion, preferably at a temperature of about 25° C. to about 150° C. Preferred crosslinking agents are ethylene glycol diglycidyl ether (EGDGE), a water-soluble diglycidyl ether, and a dibromoalkane, an alcohol-soluble compound.

The basic resin, either strongly or weakly basic, therefore, can be any resin that acts as an SAP in its charged form. The basic resin typically contains amino or guanidino moieties. Examples of basic resins include a poly(vinylamine), a polyethylenimine, a poly(vinylguanidine), a poly(allylamine), a poly(allylguanidine), or a poly (dialkylaminoalkyl (meth) acrylamide) prepared by polymerizing and lightly crosslinking a monomer having the structure 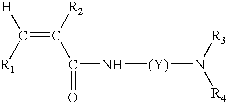
or its ester analog 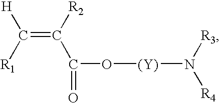
wherein R₁ and R₂, independently, are selected from the group consisting of hydrogen and methyl, Y is a divalent straight chain or branched organic radical having 1 to 8 carbon atoms, and R₃ and R₄, independently, are alkyl radicals having 1 to 4 carbon atoms. Preferred basic resins include a poly(vinylamine), polyethylenimine, poly(vinylguanadine), poly(dimethylaminoethyl acrylamide) (poly(DAEA)), and poly(dimethylaminopropyl methacrylamide) (poly(DMAPMA)). Analogous to microdomains of an acidic resin, the present multicomponent SAPs can contain microdomains of a single basic resin, microdomains containing a mixture of basic resins, or microdomains of different basic resins.

The present multicomponent SAPs can be prepared by various methods. It should be understood that the exact method of preparing a multicomponent SAP is not limited by the following embodiments. Any method that provides a particle having at least one microdomain of an acidic resin in contact with or in close proximity to at least one microdomain of a basic resin is suitable.

In one method, dry particles of a basic resin, optionally surface crosslinked and/or annealed, are admixed into a rubbery gel of an acidic resin. The resulting mixture is extruded, then dried, and optionally surface crosslinked and/or annealed, to provide multicomponent SAP particles having microdomains of a basic resin dispersed in a continuous phase of an acidic resin. Alternatively, particles of an acidic resin, optionally surface crosslinked and/or annealed, can be admixed into a rubbery gel of a basic resin, and the resulting mixture is extruded and dried, and optionally surface crosslinked and/or annealed, to provide multicomponent SAP particles having microdomains of an acidic resin dispersed in a continuous phase of a basic resin.

In another method, dry particles of an acidic resin can be admixed with dry particles of a basic resin, and the resulting mixture is formed into a hydrogel, then extruded, to form multicomponent SAP particles.

In yet another method, a rubbery gel of an acidic resin and a rubbery gel of a basic resin, each optionally surface crosslinked and/or annealed, are coextruded, and the coextruded product is dried, and optionally surface crosslinked and/or annealed, to form multicomponent SAP particles containing microdomains of the acidic resin and the basic resin dispersed throughout the particle.

The method of preparing the present multicomponent SAP particles, therefore, is not limited, and does not require an extrusion step. Persons skilled in the art are aware of other methods of preparation wherein the multicomponent SAP contains at least one microdomain of an acidic resin and at least one microdomain of a basic resin in contact or in close proximity with each other. One example is agglomeration of fine particles of at least one acidic resin and at least one basic resin with each other, and optionally a matrix resin, to provide a multicomponent SAP particle containing microdomains of an acidic and/or basic resin. The multicomponent SAP particles can be ground to a desired particle size, or can be prepared by techniques that yield the desired particle size. Other nonlimiting methods of preparing an SAP particle of the present invention are set forth in the examples.

In embodiments wherein an acidic resin and a basic resin are present as microdomains within a matrix of a matrix resin, particles of an acidic resin and a basic resin are admixed with a rubbery gel of a matrix resin, and the resulting mixture is extruded, then dried, to form multicomponent SAP particles having microdomains of an acidic resin and a basic resin dispersed in a continuous phase of a matrix resin. Alternatively, rubbery gels of an acidic resin, basic resin, and matrix resin can be coextruded to provide a multicomponent SAP containing microdomains of an acidic resin, a basic resin, and a matrix resin dispersed throughout the particle. In this embodiment, the acidic resin, basic resin, and resulting multicomponent SAP, each can be optionally surface crosslinked and/or annealed.

The matrix resin is any resin that allows fluid transport such that a liquid medium can contact the acidic and basic resin. The matrix resin typically is a hydrophilic resin capable of absorbing water. Nonlimiting examples of matrix resins include poly(vinyl alcohol), poly(N-vinylformamide), polyethylene oxide, poly(meth)acrylamide, poly(hydroxyethyl acrylate), hydroxyethylcellulose, methylcellulose, and mixtures thereof. The matrix resin also can be a conventional water-absorbing resin, for example, a polyacrylic acid neutralized greater than 50 mole %, and typically greater than 60 mole %.

In preferred embodiments, the acidic resin, the asic resin, and/or the multicomponent SAP particles are surface treated and/or annealed. Surface treatment and/or annealing results in surface crosslinking of the particle. In especially preferred embodiments, the acidic and/or basic resins comprising the multicomponent SAP particles are surface treated and/or annealed, and the entire multicomponent SAP particle is surface treated and/or annealed. It has been found that surface treating and/or annealing of an acidic resin, a basic resin, and/or a multicomponent SAP article of the present invention enhances the ability of the resin or multicomponent SAP particle to absorb and retain aqueous media under a load.

Surface crosslinking is achieved by contacting an acidic resin, a basic resin, and/or a multicomponent SAP particle with a solution of a surface crosslinking agent to wet predominantly only the outer surfaces of the resin or SAP particle. Surface crosslinking and drying of the resin or multicomponent SAP particle then is performed, preferably by heating at least the wetted surfaces of the resin or multicomponent SAP particles.

Typically, the resins and/or SAP particles are surface treated with a solution of a surface crosslinking agent. The solution contains about 0.01% to about 4%, by weight, surface crosslinking agent, and preferably about 0.4% to about 2%, by weight, surface crosslinking agent in a suitable solvent, for example, water or an alcohol. The solution can be applied as a fine spray onto the surface of freely tumbling resin particles or multicomponent SAP particles at a ratio of about 1:0.01 to about 1:0.5 parts by weight resin or SAP particles to solution of surface crosslinking agent. The surface crosslinker is present in an amount of 0% to about 5%, by weight of the resin or SAP particle, and preferably 0% to about 0.5% by weight. To achieve the full advantage of the present invention, the surface crosslinker is present in an amount of about 0.001% to about 0.1% by weight.

The crosslinking reaction and drying of the surface-treated resin or multicomponent SAP particles are achieved by heating the surface-treated polymer at a suitable temperature, e.g., about 25° C. to about 150° C., and preferably about 105° C. to about 120° C. However, any other method of reacting the crosslinking agent to achieve surface crosslinking of the resin or multicomponent SAP particles, and any other method of drying the resin or multicomponent SAP particles, such as microwave energy, or the such as, can be used.

With respect to the basic resin, or multicomponent SAP particles having a basic resin present on the exterior surface of the particles, suitable surface crosslinking agents include di- or polyfunctional molecules capable of reacting with amino groups and crosslinking a basic resin. Preferably, the surface crosslinking agent is alcohol or water soluble and possesses sufficient reactivity with a basic resin such that crosslinking occurs in a controlled fashion at a temperature of about 25° C. to about 150° C.

Nonlimiting examples of suitable surface crosslinking agents for basic resins include:
(a) dihalides and disulfonate esters, for example, compounds of the formula
   Y—(CH₂)ₚ—Y,
   wherein p is a number from 2 to 12, and Y, independently, is halo (preferably bromo), tosylate, mesylate, or other alkyl or aryl sulfonate esters;
(b) multifunctional aziridines;
(c) multifunctional aldehydes, for example, glutaraldehyde, trioxane, Paraformaldehyde, terephthaldehyde, malonaldehyde, and glyoxal, and acetals and bisulfites thereof;
(d) halohydrins, such as epichlorohydrin;
(e) multifunctional epoxy compounds, for example, ethylene glycol diglycidyl ether, bisphenol A diglycidyl ether, and bisphenol F diglycidyl ether,
(f) multifunctional carboxylic acids and esters, acid chlorides, and anhydrides derived therefrom, for example, di- and polycarboxylic acids containing 2 to 12 carbon atoms, and the methyl and ethyl esters, acid chlorides, and anhydrides derived therefrom, such as oxalic acid, adipic acid, succinic acid, dodecanoic acid, malonic acid, and glutaric acid, and esters, anhydrides, and acid chlorides derived therefrom;
(g) organic titanates, such as TYZOR AA, available from E.I. DuPont de Nemours, Wilmington, Del.;
(h) melamine resins, such as the CYMEL resins available from Cytec Industries, Wayne, N.J.;
(i) hydroxymethyl ureas, such as N,N′-dihydroxymethyl-4,5-dihydroxyethylene urea;
(j) multifunctional isocyanates, such as toluene diisocyanate, isophorone diisocyanate, methylene diisocyanate, xylene diisocyanate, and hexamethylene diisocyanate; and
(k) other crosslinking agents for basic water-absorbing resins known to persons skilled in the art.

A preferred surface crosslinking agent is a dihaloalkane, ethylene glycol diglycidyl ether (EGDGE), or a mixture thereof, which crosslink a basic resin at a temperature of about 25° C. to about 150° C. Especially preferred surface crosslinking agents are dibromoalkanes containing 3 to 10 carbon atoms and EGDGE.

With respect to the acidic water-absorbing resins, or multicomponent SAP particles having an acidic resin on the exterior surface of the particles, suitable surface crosslinking agents are capable of reacting with acid moieties and crosslinking the acidic resin. Preferably, the surface crosslinking agent is alcohol soluble or water soluble, and possesses sufficient reactivity with an acidic resin such that crosslinking occurs in a controlled fashion, preferably at a temperature of about 25° C. to about 150° C.

Nonlimiting examples of suitable surface crosslinking agents for acidic resins include:
(a) polyhydroxy compounds, such as glycols and glycerol;
(b) metal salts;
(c) quaternary ammonium compounds;
(d) a multifunctional epoxy compound;
(e) an alkylene carbonate, such as ethylene carbonate or propylene carbonate;
(f) a polyaziridine, such as 2,2-bishydroxymethyl butanol tris[3-(1-aziridine propionate]);
(g) a haloepoxy, such as epichlorhydrin;
(h) a polyamine, such as ethylenediamine;
(i) a polyisocyanate, such as 2,4-toluene diisocyanate; and
(j) other crosslinking agents for acidic water-absorbing resins known to persons skilled in the art.

In addition to, or in lieu of, surface treating, the acidic resin, the basic resin, the matrix resin, or the entire SAP particle, or any combination thereof, can be annealed to improve water absorption and retention properties under a load. It has been found that heating a resin for a sufficient time at a sufficient temperature above the Tg (glass transition temperature) of the resin or microdomains improves the absorption properties of the resin.

In accordance with an important feature of the present invention, a strong acidic resin can be used with either a strong basic resin or a weak basic resin, or a mixture thereof. A weak acidic resin can be used with a strong basic resin or a weak basic resin, or a mixture thereof. Preferably, the acidic resin is a weak acidic resin and the basic resin is a weak basic resin. This result is unexpected in view of the ion exchange art wherein a combination of a weak acidic resin and a weak basic resin does not perform as well as other combinations, e.g., a strong acidic resin and a strong basic resin. In more preferred embodiments, the weak acidic resin, the weak basic resin, and/or the multicomponent SAP particles are surface crosslinked and/or annealed.

The following nonlimiting examples illustrate the preparation of the multicomponent SAP particles of the present invention. Additional examples illustrating the preparation of multicomponent SAP particles can be found in U.S. Pat. No. 6,222,091, incorporated herein by reference.

### EXAMPLE 1

### Preparation of Poly(Acrylic Acid) 0% Neutralized (Poly(AA) DN=0)

A monomer mixture containing acrylic acid (270 grams), deionized water (810 grams), methylenebisacrylamide (0.4 grams), sodium persulfate (0.547 grams), and 2-hydroxy-2-methyl-1-phenyl-propan-1-one (0.157 grams) was prepared, then sparged with nitrogen for 15 minutes. The monomer mixture was placed into a shallow glass dish, then the monomer mixture was polymerized under 15 mW/cm² of UV light for 25 minutes. The resulting poly(AA) was a rubbery gel.

The rubbery poly(AA) gel was cut into small pieces, then extruded through a KitchenAid Model K5SS mixer with meat grinder attachment. The extruded gel was dried in a forced-air oven at 120° C., and finally ground and sized through sieves to obtain the desired particle size.

This procedure provided a lightly crosslinked polyacrylic acid hydrogel with a degree of neutralization of zero (DN=0).

### EXAMPLE 2

### Preparation of a Poly(N-vinylformamide) and a Poly(vinylamine)

A monomer mixture containing N-vinylformamide (250 grams), deionized water (250 grams), methylenebisacrylamide (1.09) grams), and V-50 initiator (0.42 grams) was placed in a shallow dish, then polymerized under an ultraviolet lamp as set forth in Example 1 until the mixture polymerized into a rubbery gel. The lightly crosslinked poly(N-vinylformamide) then was hydrolyzed with a sodium hydroxide solution to yield a lightly crosslinked poly(vinylamine).

### EXAMPLE 3

### Preparation of a Crosslinked Poly(vinylamine) Resin

To 2 liters of a 3% by weight aqueous poly(vinylamine) solution was added 0.18 g of ethyleneglycol diglycidyl ether (EGDGE). The resulting mixture was stirred to dissolve the EGDGE, then the mixture was heated to about 60° C. and held for one hour to gel. The gel was heated to about 80° C. and held until about 90% of the water was removed. The resulting get then was extruded and dried to a constant weight at 80° C. The dried, lightly crosslinked poly(vinylamine) then was cryogenically milled to form a granular material.

### EXAMPLE 4

### Preparation of a Multicomponent SAP Having a Poly(AA) Core Surrounded by a PEI Shell

Sorbitan monooleate (0.81 g) was dissolved in 200 ml of heptane. Ten grams of crosslinked, unneutralized polyacrylic acid was added to this solution to act as seed for the core/shell composite particles. The resulting mixture was stirred at 700 rpm-with a paddle stirrer. Polyethyleneimine (PEI) (27.6 g, 30% in water, M_{w}=750,000) was added to the polyacrylic acid/heptane slurry, followed immediately by the addition of 3.6 g of EGDGE. The EGDGE and PEI were allowed to cure for 4.5 hours at room temperature. The resulting SAP particles were allowed to settle, and the supernatant heptane was decanted. The SAP particles were rinsed three times with 100 ml of acetone. The SAP particles were allowed to dry overnight at room temperature, then further dried at 80° C. for 2 hours to yield 23.43 g of the multicomponent SAP particles.

### EXAMPLE 5

### Preparation of a Multicomponent SAP Having a Poly(AA) Core Surrounded by a Poly(vinylamine) Shell

Sorbitan monooleate (1.88 g) was dissolved in 500 ml of heptane. Ten grams of crosslinked, unneutralized polyacrylic acid was added to this solution to act as seed for the core/shell composite particles. The resulting mixture was stirred at 700 rpm with a paddle stirrer. Poly(vinylamine) (84 g, 10.67% in water, M_{w}>100,000) was added to the polyacrylic acid/heptane slurry, followed immediately by the addition of 1.5 g of EGDGE. The EGDGE and poly(vinylamine) were allowed to cure for 6 hours at room temperature. The resulting SAP particles were allowed to settle, and the supernatant heptane was decanted. The SAP particles were rinsed three times with 200 ml of acetone. The SAP particles were dried at 80° C. for 3 hours to yield 17.89 g of the multicomponent SAP particles.

In another embodiment, the multicomponent SAP particles can be mixed with particles of a second water-absorbing resin to provide an SAP material A having improved absorption properties. The second water-absorbing resin can be an unneutralized acidic water-absorbing resin, an unneutral-ized basic water-absorbing resin, or a mixture thereof. Like the multicomponent SAP particles, the second water-absorbing resin particles have a particle size of about 38 to about 300 μm, and a median particle size of less than about 180 μm. The second water-absorbing resin is neutralized 0% to 50%.

SAP material A of this embodiment comprises about 10% to about 90%, and preferably about 25% to about 85%, by weight, multicomponent SAP particles and about 10% to about 90%, and preferably, about 25% to about 85%, by weight, particles of the second water-absorbing resin. More preferably, SAP material A contains about 30% to about 75%, by weight, multicomponent SAP particles. To achieve the full advantage of the present invention, SAP material A contains about 35% to about 75%, by weight, the multicomponent SAP particles. The multicomponent SAP particles can be prepared by any of the previously described methods, e.g., extrusion, agglomeration, or interpenetrating polymer network. The multicomponent SAP particles and particles of the second water-absorbing resin can be of any shape, e.g., granular, fiber, powder, or platelets.

The second water-absorbing resin can be any of the previously discussed acidic resins used in the preparation of a multicomponent SAP. A preferred acidic water-absorbing resin used as the second resin is unneutralized polyacrylic acid (PAA), e.g., DN up to about 50%. The second water-absorbing resin also can be any of the previously discussed basic resins used in the preparation of a multicomponent SAP. Preferred basic water-absorbing resins used as the second resin are unneutralized poly(vinylamine) or unneutral-ized polyethylenimine. Blends of acidic resins, or blends of basic resins, can be used as the second water-absorbing resin. Blends of an acidic resin and a basic resin also can be used as the second water-absorbing resin. The second water-absorbing resin is optionally surface crosslinked or annealed.

An example of an SAP material A comprising multicomponent SAP particles and particles of a second water-absorbing resin is a mixture of multicomponent SAP particles and unneutralized (DN=0) polyacrylic acid (PAA) particles. As used here and throughout the specification (PAA) (DN=0) refers to 100% unneutralized (PAA). The multicomponent SAP particles contain microdomains of poly(vinylamine) dispersed in (PAA)(DN=0). The poly(vinylamine)/(PAA) weight ratio of the multicomponent SAP particles is 55/45.

In yet another embodiment, a superabsorbent material B comprises an admixture of particles of an unneutralized basic water-absorbing resin, like a polyvinylamine, and particles of an unneutralized acidic water-absorbing resin, like polyacrylic acid, wherein both the acidic and basic water-absorbing resins have a particle size of about 38 to about 300 μm and a median particle size of less than about 180 μm. Both the acidic and basic water-absorbing resin are neutralized 0% to about 50%. The acidic and basic water-absorbing resins can be any of the previously discussed acidic and basic resins used in the preparation of a multicomponent SAP, and either or both are optionally surface crosslinked or annealed.

SAP material B of this embodiment comprises about 10% to about 90%, and preferably about 25% to about 85%, by weight, acidic water-absorbing resin particles and about 10% to about 90%, and preferably, about 25% to about 85%, by weight, basic water-absorbing resin particles. More preferably, SAP material B contains about 30% to about 75%, by weight, acidic resin particles. To achieve the full advantage of the present invention, SAP material B contains about 35% to about 75%, by weight, the acidic resin particles.

A preferred acidic water-absorbing resin is PAA (DN=0). Preferred basic water-absorbing resins used are an unneutralized poly(vinylamine) or an unneutralized polyethylenimine. Blends of acidic resins and/or blends of basic resins can be used in SAP material B.

An example of an SAP material B comprising particles of an acidic and a basic water-absorbing resin is a mixture of unneutralized (DN=0) PAA particles and unneutralized poly(vinylamine) (PVAm). The PVAm/PAA weight ratio of the SAP material B is 30/70.

Superabsorbent materials A and B containing small size acidic resin and basic resin particles demonstrates unexpected water absorption and retention properties. Such SAP materials comprise two uncharged, slightly crosslinked polymers. When contacted with water or an aqueous electrolyte-containing medium, the two uncharged resins neutralize each other to form a superabsorbent material. This also reduces the electrolyte content of the medium absorbed by polymer, further enhancing the polyelectrolyte effect. Neither polymer in its uncharged form behaves as an SAP by itself when contacted with a fluid. However, a superabsorbent material B, which contains a simple mixture of two resins, one acidic and one basic, is capable of acting as an absorbent material because the two resins are converted to their polyelectrolyte form. Prior superabsorbent mixed bed systems have demonstrated good water absorption and retention properties. However, the present SAP material B, containing small particle size resins, exhibit improved water absorption and retention, and improved permeability, over mixtures of acidic resin particles and basic resin particles having a larger particle size.

In the test results set forth below, the multicomponent SAP particles of the present invention were tested for absorption under no load (AUNL) and absorption under load at 0.28 psi and 0.7 psi (AUL (0.28 psi) and AUL (0.7 psi)). Absorption under load (AUL) is a measure of the ability of an SAP to absorb fluid under an applied pressure. The AUL was determined by the following method, as disclosed in U.S. Pat. No. 5,149,335, incorporated herein by reference.

An SAP (0.160 g+/−0.001 g) is carefully scattered onto a 140-micron, water-permeable mesh attached to the base of a hollow Plexiglas cylinder with an internal diameter of 25 mm. The sample is covered with a 100 g cover plate and the cylinder assembly weighed. This gives an applied pressure of 20 g/cm² (0.28 psi). Alternatively, the sample can be covered with a 250 g cover plate to give an applied pressure of 51 g/cm² (0.7 psi). The screened base of the cylinder is placed in a 100 mm petri dish containing 25 milliliters of a test solution (usually 0.9% saline), and the polymer is allowed to absorb for 1 hour (or 3 hours). By re-weighing the cylinder assembly, the AUL (at a given pressure) is calculated by dividing the weight of liquid absorbed by the dry weight of polymer before liquid contact.

In addition to an ability to absorb and retain relatively large amounts of a liquid, it also is important for an SAP to exhibit good permeability, and, therefore, rapidly absorb the liquid. Therefore, in addition to absorbent capacity, or gel volume, useful SAP particles also have a high gel strength, i.e., the particles do not deform after absorbing a liquid. In addition, the permeability or flow conductivity of a hydrogel formed when SAP particles swell, or have already swelled, in the presence of a liquid is extremely important property for practical use of the SAP particles. Differences in permeability or flow conductivity of the absorbent polymer can directly impact on the ability of an absorbent article to acquire and distribute body fluids.

Many types of SAP particles exhibit gel blocking. “Gel blocking” occurs when the SAP particles are wetted and swell, which inhibits fluid transmission to the interior of the SAP particles and between absorbent SAP particles. Gel blocking can be a particularly acute problem if the SAP particles lack adequate gel strength, and deform or spread under stress after the SAP particles swell with absorbed fluid.

Accordingly, an SAP particle can have a satisfactory AUL value, but will have inadequate permeability or flow conductivity to be useful at high concentrations in absorbent structures. In order to have a high AUL value, it is only necessary that the hydrogel formed from the SAP particles has a minimal permeability such that, under a confining pressure of 0.3 psi, gel blocking does not occur to any significant degree. The degree of permeability needed to simply avoid gel blocking is much less than the permeability needed to provide good fluid transport properties. Accordingly, SAPs that avoid gel blocking and have a satisfactory AUL value can still be greatly deficient in these other fluid handling properties.

An important characteristic of the small-size SAP particles of the present invention is permeability when swollen with a liquid to form a hydrogel zone or layer, as defined by the Saline Flow Conductivity (SFC) value of the SAP particles. SFC measures the ability of an SAP to transport saline fluids, such as the ability of the hydrogel layer formed from the swollen SAP to transport body fluids. A material having relatively high SFC value is an air-laid web of wood pulp fibers. Typically, an air-laid web of pulp fibers (e.g., having a density of 0.15 g/cc) exhibits an SFC value of about 200×10⁻⁷ cm³sec/g. In contrast., typical hydrogel-forming SAPs exhibit SFC values of 1×10⁻⁷ cm³sec/g or less. When an SAP is present at high concentrations in an absorbent structure, and then swells to form a hydrogel under usage pressures, the boundaries of the hydrogel come into contact, and interstitial voids in this high SAP concentration region become generally bounded by hydrogel. When this occurs, the permeability or saline flow conductivity properties in this region is generally indicative of the permeability or saline flow conductivity properties of a hydrogel zone formed from the SAP alone. Increasing the permeability of these swollen high concentration regions to levels that approach or even exceed conventional acquisition/distribution materials, such as wood pulp fluff, can provide superior fluid handling properties for the absorbent structure, thus decreasing incidents of leakage, especially at high fluid loadings.

Accordingly, it would be highly desirable to provide SAP particles having an SFC value that approaches or exceeds the SFC value of an air-laid web of wood pulp fibers. This is particularly true if high, localized concentrations of SAP particles are to be effectively used in an absorbent article. High SFC values also indicate an ability of the resultant hydrogel to absorb and retain body fluids under normal usage conditions. A method for determining the SFC value of SAP particles is set forth in Goldman et al. U.S. Pat. No. 5,599,335, incorporated herein by reference.

The small-size SAP particles of the present invention show a substantial improvement in AUL at 0.7 psi and SFC. Accordingly, a present small particle size multicomponent SAP particle has an SFC value of at least about 20 ×10⁻⁷ cm³sec/g, and preferably at least about 50×10⁻⁷ cm³sec/g. To achieve the full advantage of the present invention, the SFC value is at least about 100×10⁻⁷ cm³sec/g, and can range up to about 2000×10⁻⁷ cm³sec/g.

In the following discussion and in FIGS. 4-10, the term “SAF” is defined as a multicomponent SAP containing 55 wt % lightly crosslinked PAA (DN=0) and 45% wt % lightly crosslinked, unneutralized poly(vinylamine) (PVAm, DN=0). The term “LAF” is defined as a multicomponent SAP containing 70 wt % PAA (DN=0) and 30 wt % PVAm (DN=0). The term 70). Particle size of the superabsorbent particles is given in microns (μm).

The following table illustrates the SFC values (×10⁻⁷ cm³ sec/g), i.e., an SFC unit for SAF and LAF:

| | Particle | | SFC | No. of | Std. |
|---|---|---|---|---|---|
| | Size Range | | (avg.) | Replicate Tests | Dev. |
| SAF | <180 | μm | 941 | 6 | 647 |
| SAF | 105-180 | μm | 710 | 2 | 308 |
| SAF | 75-105 | μm | 1591 | 2 | 431 |
| SAF | <75 | μm | 1991 | 2 | 1258 |
| SAF | <180 | μm | 1453 | 6 | 635 |
| SAF | 105-180 | μm | 1358 | 2 | 169 |
| SAF | 75-105 | μm | 1413 | 2 | 245 |
| SAF | <75 | μm | 1732 | 2 | 503 |
| LAF | <180 | μm | 20 | 2 | 19 |
| LAF | 105-180 | μm | 40 | 2 | 2 |
| LAF | 75-105 | μm | 9 | 2 | 10 |
| LAF | <75 | μm | 47 | 2 | 24 |
| A2300 | 180-710 | μm | 30-50 | | |
| A2300 | <180 | μm | 0 | 2 | 0 |
| A2300 | 105-180 | μm | 0 | 2 | 0 |
| A2300 | 75-105 | μm | 0 | 2 | 0 |
| A2300 | <75 | μm | 0 | 2 | 0 |

The results in the above table were determined as follows. The results evaluate the performance of small particle size multicomponent superabsorbent particles in synthetic urine and plasmion (synthetic plasma) for absorbency (AUL and AUNL) and fluid flow (SFC).

Procedure

Small particle size multicomponent superabsorbent particles were separated by particle size into the following ranges <180 μm, 105-180, 75-105, and <75. AUL, AUNL, and SFC values then were determined on each of the above particle size ranges. Two standard formulations (SAF) and one low amine formulation (LAF) of multicomponent superabsorbent particles were evaluated. In addition, a sample of standard, commercial A2300 SAP was evaluated as a control.

Results—SAF

Synthetic Urine: The two different samples of SAF performed identically in all AUL tests. As particle size decreased, the AUL (0.7 psi) decreased merely about 8% (from about 47 g/g to about 43 g/g). These results were about three times better than the A2300 control of the same particle size (i.e., 15 g/g), and about 1.5 to 2 times better than commercial sized A2300 (28 g/g). The AUNL values were more variable, and did not show a clear trend (i.e., about 57 to 62 g/g).

Synthetic Plasma: Similarly, the two SAF batches were very similar in performance. The downward trend with particle size for the AUL (0.7 psi) load performance was about a 4 to 5% decrease (from about 32 g/g to 30 g/g), which is negligible. The multicomponent superabsorbent particle results were about 2.5 times better than the control A2300 results, which were about 13 g/g for small particle sized material and about 14 g/g for commercial sized A2300. The AUNL values did not show a clear trend, with results ranging from 47 to 56 g/g.

SFC: The SFC values varied, even within sample repeats. While no clear trend was apparent, the results consistently were greater than 150 SFC units, and were as high as 1250 SFC units. The average value was about 500 SFC units. The control A2300 showed no flow for all of the fine particle sizes, i.e., SFC=0.

Results—LAF

Synthetic Urine: The AUL (0.7 psi) performance showed a substantial drop compared to the standard sized (180-710 μm) multicomponent superabsorbent particles. AUL values decreased about 35% from 34 g/g at particle size<180 μm down to 22 g/g at particle size<75 μm. While the small particle sized LAF were 1.5-2.3 times better than the A2300 small particle size particles, particle size cuts less than 105 μm were not as good as commercial sized A2300. AUNL values showed no clear trend, being about 64 g/g on average.

Synthetic Plasma: A decrease in AUL (0.7 psi) values of about 14% was observed with decreasing particle size (i.e., from 28 g/g down to 23 g/g). These values were about two times better than the small particle size A2300 and commercial size A2300. AUNL values had no clear trend, having an average value of about 55 g/g.

SFC: The SFC data for the LAF was inferior to the SFC data for the SAF. While variable, the average LAF SFC value was about 30 SFC units. This data was substantially better than the SFC values for A2300 control SAP, but not as high as the data for the SAF (500 SFC units).

An SAP material A or B has an SFC of greater than 15×10⁻⁷ cm³ sec/g, and typically greater than 20×10⁻⁷ cm³ sec/g. Preferred embodiments have an SFC about 30×10⁻⁷ cm³ sec/g or greater, for example, up to about 800×10⁻⁷ cm³ sec/g.

In another test, the free swell rate (FSR) of a present multicomponent SAP particle, or an SAP material A or B, was determined. The FSR test, also known as a lockup test, is well known to persons skilled in the art. The present multicomponent SAP particles, or an SAP material A or B, have an FSR (in g/g/sec) of greater than 0.35, preferably greater than 0.40, and most preferably greater than 0.45. These FSR values further show the improved ability of the present small size SAP particles to absorb and retain larger amounts of an electrolyte-containing liquid quickly.

The small particle size superabsorbent polymer particles of the present invention are useful in hygienic products, such as diapers, adult incontinence articles, feminine napkins, general purpose wipes and cloths, and in aqueous waste solidification. In accordance with an important feature of the present invention, the hygienic product, or other absorbent article, has a core containing about 50% to 100%, preferably about 60 to 100%, more preferably about 75 to 100%, and most preferably about 75% to 95% of a small particle size multicomponent SAP, or an SAP mixture A or B.

Multicomponent SAP particles and mixed beds of resins have been used in diaper cores in high amounts, and exhibit excellent acquisition rates, but the rewet values can be very high. This observation is attributed to the open nature of the core after the first hydration, or insult, which causes a loss of capillary action in the core. The open nature of the core after the first hydration is due to particle/particle and particle/fluff adhesion in the core. As the particles swell, these adhesive forces cause the formation of large voids that are incapable of capillary fluid transport.

The present invention utilizes a small particle size multicomponent superabsorbent particles, or a mixed bed of small particle size resins (superabsorbent materials A and B), to maintain capillary wicking action in low fluff and fluffless cores. Small particle size SAP particles have an inherent wicking (i.e., capillary) action. Normally, a conventional SAP is used at larger particle sizes (e.g., >400 μm) because the hydrating SAP is subject to gel blocking. However, because ion exchanging SAPs can have excellent gel bed permeabilities (i.e., high SFC) even at very small particle sizes, smaller particle size ranges can be used in low fluff cores. With a sufficiently small SAP particle size, the wicking action is sufficient to allow the complete elimination of the cellulosic fiber. The small particle size multicomponent SAP, or mixed bed superabsorbent materials A and B, is capable of performing both the wicking and storage functions of a core.

The absorbent cores of the present invention can range from heavily loaded cores (e.g., 60-95 wt % superabsorbent polymer/5-40 wt % fluff) to fluffless cores (i.e., 100% SAP). The fluffless cores typically are constructed of alternate layers of (a) tissue and (b) multicomponent superabsorbent particles, or SAP materials A or B, having a median particle size of less than 180 μm. Additionally, a top, or acquisition, layer of standard particle size superabsorbent polymer (i.e., particle size range of about 170 μm to about 800 μm) optionally can be used to provide faster acquisition rates. The present invention also eliminates the problem of horizontal expansion of the core.

Present day diapers generally consist of a top-sheet made from a nonwoven material that is in contact with the skin of the wearer, an acquisition layer below (i.e., opposite the skin of wearer) the topsheet, a core that is below the acquisition layer, and a backsheet below the core. This construction is well known in the industry. In a preferred embodiment, the present diaper consists essentially of a topsheet, a core, and a backsheet, i.e., an acquisition layer is not present. As illustrated below, the improvements provided by the present small particle size multicomponent SAP particles, or superabsorbent materials A or B, permit an acquisition layer to be omitted from a disposable diaper. Such a result is important in the art because an expensive acquisition layer can be omitted, the diaper is lighter and thinner, and absorptive properties are not adversely affected.

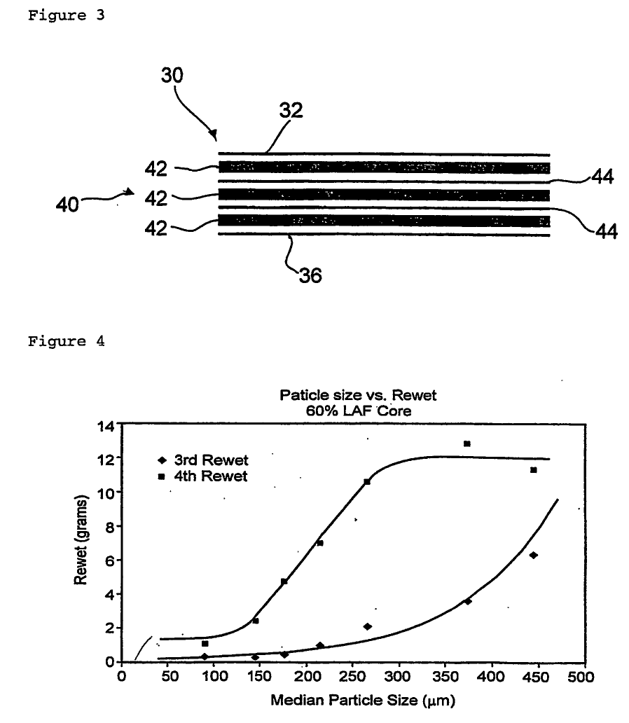

A fluffless core of the present invention is illustrated in FIG. 3. FIG. 3 shows a cross section of an aborbent article 30 having a topsheet 32, a backsheet 36, and an absorbent core indicated by 40 positioned between top-sheet 32 and backsheet 36. As shown in FIG. 3, core 40 comprises a plurality of layers 42. Layers 42 comprise small article size SAP particles, and are separated from one another by tissue layers 44.

The fluffless core in FIG. 3 can include additional layer and tissue layer (not shown) disposed between topsheet 32 and layer 42. This optional additional layer serves as an acquisition/distribution layer and contains a conventional SAP, e.g., PAA (DN=70) having a particle size range of about 170 μm to 800 μm. A fluffless core illustrated in FIG. 3 can contain one to five, and preferably two to four layers 42, i.e., one to five layers of small particle size SAP particles.

To further illustrate that the present small particle size multicomponent SAP particles and superabsorbent materials A and B (a) have an improved ability to absorb liquids faster, (b) have a better liquid diffusion rate, and (c) have an improved ability to absorb and retain liquids, laboratory diaper cores containing the present multicomponent SAP particles were prepared and compared to laboratory diaper cores containing a conventional SAP.

Cores referred to as “fluffless” cores contain 100% of an SAP and are free of cellulosic fibers or other “fluff” materials. Typically, high loading commercial diapers contain 45% to 60% by weight of a cellulosic fibers to achieve rapid absorption of a liquid.

For an absorbent article having a core containing a “fluff” component, the “fluff” component comprises a fibrous material in the form of a web or matrix. Fibers useful in the present invention include naturally occurring fibers (modified or unmodified), as well as synthetic fibers. Examples of suitable unmodified/modified naturally occurring fibers include cotton, Esparto grass, bagasse, kemp, flax, silk, wool, wood pulp, chemically modified wood pulp, jute, rayon, ethylcellulose, and cellulose acetate. Suitable synthetic fibers can be made from polyvinyl chloride, polyvinyl fluoride, polytetrafluoroethylene, polyvinylidene chloride, polyacrylics such as ORLON®, polyvinyl acetate, polyethylvinyl acetate, nonsoluble or soluble polyvinyl alcohol, polyolefins such as polyethylene (e.g., PULPEX®) and polypropylene, polyamides (e.g., nylon), polyesters (e.g., DACRON® or KODEL®), polyurethanes, polystyrenes, and the like. The fibers can comprise solely naturally occurring fibers, solely synthetic fibers, or any compatible combination of naturally occurring and synthetic fibers.

Hydrophilic fibers are preferred, and include cellulosic fibers, modified cellulosic fibers, rayon, polyester fibers, such as polyethylene terephthalate (e.g., DACRON®), hydrophilic nylon (HYDROFIL), and the like. Suitable hydrophilic fibers can also be obtained by hydrophilizing hydrophobic fibers, such as surfactant-treated or silica-treated thermoplastic fibers derived from, for example, polyolefins, such as polyethylene or polypropylene, polyacrylics, polyamides, polystyrenes, polyurethanes, and the like. For reasons of availability and cost, cellulosic fibers, in particular wood pulp fibers, are preferred for use in the present invention. See WO 98/37149, incorporated herein by reference, for a complete discussion of “fluff” components for use in an absorbent article.

In general, the cores referred to herein were prepared using a conventional laboratory procedure as follows:

A laboratory core-forming unit comprising a two-chamber vacuum system forms an airlaid fluff pulp-absorbent composite matrix to produce a 12 cm×21 cm diaper core. The core-forming unit comprises a roller brush on a variable-speed laboratory motor, a fiber distribution screen in close proximity to the brush, a forming screen on an adjustable damper, and a vacuum system capable of supplying a consistent and continuous negative pressure between 8 and 15 inches of water.

The core-forming unit is contained such that the vacuum pulls the fibers and granular material from an adjustable introduction slide, through the rotating brush and distribution screen, directly onto the forming screen. The vacuum exhaust is recirculated through the inlet of the formation slide, thereby controlling the temperature and humidity of the operation.

When forming a core, the desired amount of defiberized fluff pulp is evenly disbursed in small pieces onto the brush roller in the upper chamber. In the lower chamber, a rectangular tissue, or topsheet (21 cm×12 cm), is placed onto the forming screen. For most cores, the sliding upper chamber lid is partially closed to leave about a one-half inch gap. In the case of a homogeneous pulp/SAP core, the SAP is sprinkled through the gap into the upper chamber immediately after the brush begins rotating. In order to achieve a homogeneous distribution, a small amount of SAP is added to the fluff prior to beginning the motor. The amount of time used to introduce the remainder of the SAP varies with the amount of fluff pulp utilized. After the fiber and absorbent polymer material are deposited, the motor is turned off, and the damper unit containing the laboratory core is removed from the lower chamber. The uncompressed core then is placed on a backsheet made from a polymeric film, and put into a compression unit. At this time, another rectangular tissue and a nonwoven coverstock is placed on top of the core. Absorbent cores are compressed for a given amount of time, typically 5 minutes, with a hydraulic press at pressures of between about 5,000 pounds and about 10,000 pounds, and typically about 7,000 pounds, to achieve the desired density. After the 5 minutes, the laboratory-prepared absorbent cores are removed from the press, weighed, and measured for thickness.

In particular, the diaper cores were prepared as follows:

(a) Low fluff cores: The fluff and small particle size multicomponent SAP particles are admixed and introduced into in the pad core forming machine in the relative amounts desired. Top and bottom tissues are placed on opposing surfaces of the core, then the core is compressed at 10,000 pounds pressure (260 psi) for five minutes.

(b) Fluffless Cores: Three grams of small particle size multicomponent SAP particles are spread out on a single tissue. A second tissue then is placed over the SAP particles, and a second three grams of small particle size multicomponent SAP particles is spread on the second tissue. A third tissue is placed on the second three grams of SAP particles, then three grams of a conventional multicomponent SAP (particle size of 180-710 μm) is spread on the third tissue. Finally, a fourth tissue is placed over the conventional multicomponent SAP. The resulting core then is compressed as in (a) above.

The cores were tested for rewet under a 0.7 psi load, liquid acquisition time, and liquid acquisition rate. The following describes the procedures to determine the acquisition and rewet under load of a hygienic article, like a diaper. These tests exhibit the rate of absorption and fluid retention of a 0.9%, by weight, saline solution, by a hygienic article over 3 to 5 separate fluid insults while under a load of 0.7 psi.

Apparatus:

100 ml separatory funnel, configured to deliver a flow rate of 7 ml/sec., or equivalent;
3.642 kg circular weight (0.7 psi) 10 cm diameter, with 2.38 cm ID perspex dose tube through the center of weight;
VWR Scientific, 9 cm filter paper or equivalent;
2.5 kg circular weight (0.7 psi)—8 cm diameter;
Digital timer;
Electronic balance (accuracy of a 0.01 gram);
Stopwatch.
   Procedure:
   1. Preparation
(a) Record the weight (g) of the hygienic article, e.g., diaper, to be tested;
(b) Place hygienic article flat on the bench top, for example, by removing any elastics and/or taping the ends of the article to the bench top;
(c) Place the 3.64 kg circular weight onto the hygienic article with the opening of the perspex dose tube positioned at the insult point (i.e., 5 cm toward the front from the center).
   2. Primary Insult and Rewet Test
(a) Measure 100 ml of 0.9% NaCl solution (i.e., 0.9% by weight sodium chloride in deionized or distilled water) into separatory funnel. Dispense the NaCl solution into the perspex tube of the weight at a flow rate of 7 ml/sec and start the timer immediately. Stop the timer when all of the NaCl solution has completely disappeared from the surface of the hygienic article at the base of the perspex tube. Record this time as the primary acquisition time (sec).
(b) After 10 minutes have elapsed, remove the weight and conduct the rewet test procedure:
   (i) Weigh a stack of 10 filter papers, record this value (dry weight).
   (ii) Place the filter papers over the insult point on the hygienic article. Set the timer for 2 minutes. Place the 2.5 kg weight onto the filter papers and start timer immediately.
   (iii) After 2 minutes have elapsed, remove the weight and reweigh the filter papers (wet weight). Subtract the dry weight of the filter papers from the wet weight, this is the rewet value. Record this value as the primary rewet value (g).
      3. Secondary Insult and Rewet Test
(a) Place the 3.64 kg weight back onto the hygienic article in the same position as before. Repeat step 2a using 50 ml NaCl solution (recoding the absorption time as the secondary acquisition time) and steps 2b (i)-(iii) using 20 filter paper (recording the rewet values as the secondary rewet).
   4. Tertiary, and Additional, Insult and Rewet Tests
(a) Place the load back onto the diaper in the same position as before. Repeat step 2a using 50 ml of NaCl solution (recording the absorption time as the tertiary acquisition time) and steps 2b (i)-(iii) using 30 filter paper (recording the rewet value as the tertiary or subsequent rewet).

The following FIGS. 4-10 illustrate improved cores and diapers that contain small particle size multicomponent SAP particles of the present invention.

FIG. 4 is a plot of rewet (grams) vs. median particle size (μm) for a diaper core containing 60%, by weight, LAF and 40%, by weight, of fluff. FIG. 4 illustrates the improved wicking observed using smaller particle size SAP particles. In particular, the fourth insult test shows a substantial improvement (i.e., drop in rewet) at 300 μm median particle size and less.

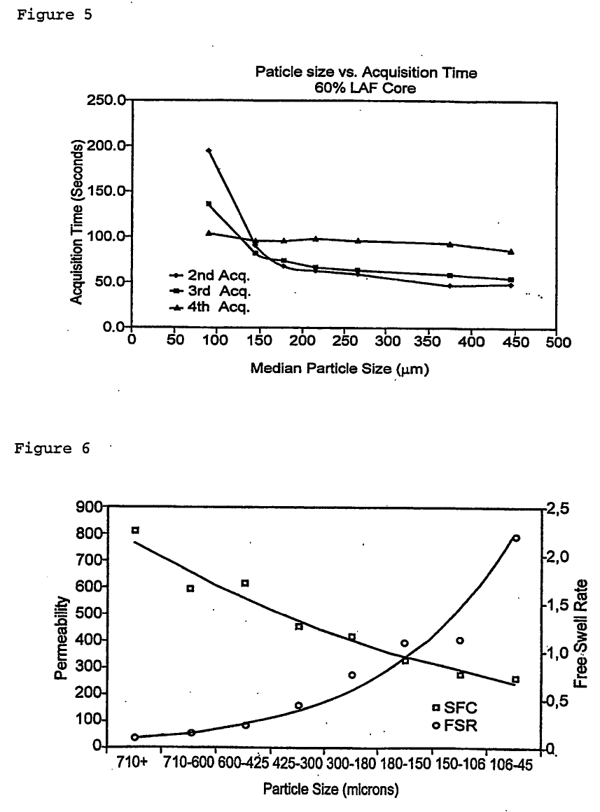

Using the same 60% LAF/40% fluff core described above, the acquisition time for a second through fourth insult test was measured. The results are illustrated in FIG. 5 showing that the acquisition time for the fourth insult is impacted slightly compared to the second and third insult acquisition time. The fourth insult acquisition time is improved over the median particle size range of 100 to 150 μm over the second and third insult acquisition times.

The 60% LAF/40% fluff core also was tested for an ability to absorb a fluid under no load (AUNL) and under a load (AUL) (0.7 psi). The following table summarizes the AUNL and AUL data after four hours for the indicated test fluid and indicated median particle size of the multicomponent SAP particles.

| | | |
|---|---|---|
| Particle Size | AUNL (g/g) | 0.7 psi AUL (g/g) |

Synthetic Urine

| | | |
|---|---|---|
| <180 μm | 62 | 47 |
| 105-180 | 60 | 47 |
| 75-105 | 61 | 44 |
| <75 | 58 | 42 |

Synthetic Plasma

| | | |
|---|---|---|
| <180 μm | 56 | 36 |
| 105-180 | 44 | 35 |
| 75-105 | 52 | 45 |
| <75 | 50 | 37 |

FIG. 6 illustrates the effects of particle size on permeability (SFC) and free swell rate (FSR). The test data in FIG. 6 was obtained using multicomponent SAP particles containing 50 wt % PAA (DN=0) and 50 wt % PVAm (DN=0). The plots in FIG. 6 show that multicomponent SAP particles having a particle size of 45 to 300 microns has an improved free swell rate compared to typically sized SAP particles of 425-710 μm. For example, a conventional PAA (DN=70) has an FSR of 0.32 which is substantially below the FSR of about 0.75 for multicomponent SAP particles having a particle size range of 45 to 106 μm. The SFC of the multicomponent SAP particles decreases with decreased particle size, but the observed SFC range of about 250 to about 400 cm³sec/g is considered excellent compared to conventional SAP particles, having an SFC of about 1×10⁻⁷ cm³sec/g or less.

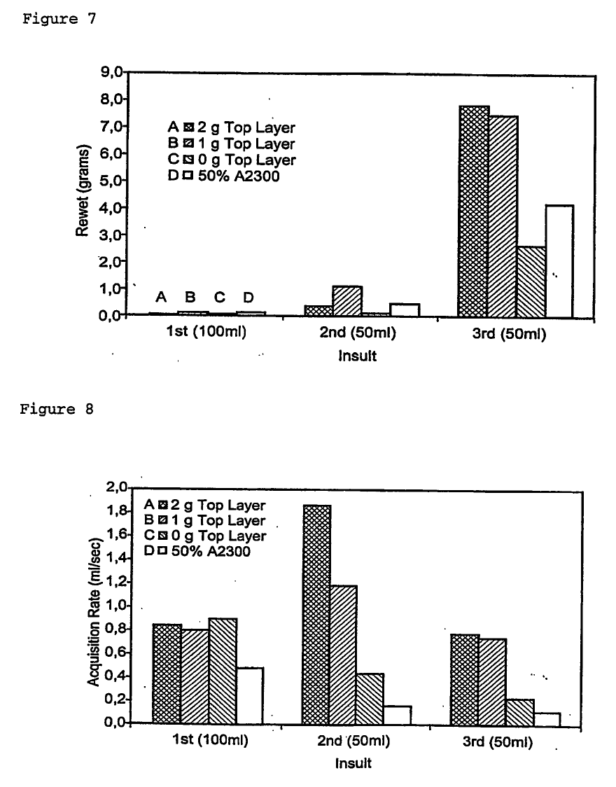

FIG. 7 contains bar graphs for rewet vs. first through third insult tests for diaper cores free of fluff. Three of the fluffless cores contain LAF multicomponent SAP particles of median particle size 170 μm. Two of these cores have an acquisition layer containing either two grams (Core A) or one gram (Core B) of LAF (particle size 300 to 710 μm). The fourth core (Core D) is a comparative core containing A2300 (DN=50).

FIG. 7 shows similar results for all four samples over the first and second insult. However, the third insult test shows a definite improvement in rewet for the core lacking an acquisition layer (Core C) compared to Cores A and B and to comparative Core D. The high rewet for Cores A and B in the third insult test is attributed to a less efficient utilization of the SAP in the acquisition layer.

FIG. 8 contains bar graphs for acquisition rate vs. first through third insult tests for Cores A-D. FIG. 8 shows a slower acquisition rate for Core C lacking an acquisition layer compared to Cores A and B having an acquisition layer. However, Cores A-C all have an improved acquisition rate compared to comparative Core D. In combination, FIGS. 7 and 8 show that a fluffless core containing a small particle size multicomponent SAP particle, either with our without an acquisition layer, outperforms a fluffless diaper cores containing PAA (DN=50).

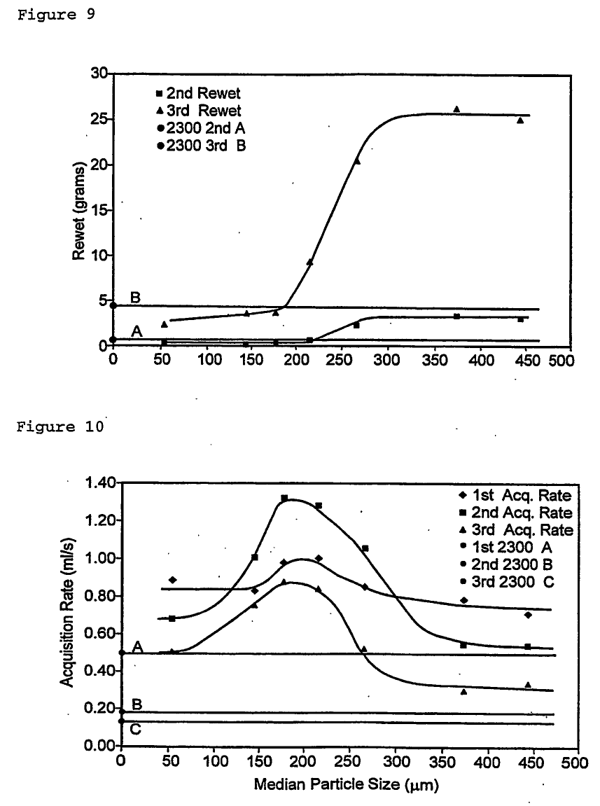

FIG. 9 contains graphs of rewet vs. median particle size for a core containing two layers of SAF, at three grams per layer, and an acquisition layer containing three grams of SAF of particle size 180-710 μm. A comparative core of identical structure but containing 50% A2300 and 50% fluff also was tested. FIG. 9 shows an improved rewet for the second and third insult tests using 0.9% aqueous sodium chloride for a median particle size of 50 to about 220 μm, compared to A2300 (DN=70) and particle size of about 180 to about 710 μm.

FIG. 10 contains graphs of acquisition rate vs. median particle size for identical cores tested in FIG. 9. FIG. 10 shows an increased, but acceptable, acquisition rate over a median particle size of 50 to about 300 μm. In all tests, the acquisition rates were improved over A2300.

Overall, the data presented in FIGS. 4-10 demonstrate that a diaper core containing small particle size particles of a multicomponent SAP, or an SAP material A or B, demonstrate excellent rewet values and acceptable to excellent acquisition rates. The practical result of these improved properties is a core having a greatly improved ability to prevent leakage in gush situations and in rewet situations, even in the absence of an acquisition layer.

The improved results demonstrated by a core of the present invention permit the thickness of the core to be reduced. Typically, cores contain 50% or more fluff or pulp to achieve rapid liquid absorption while avoiding problems like gel blocking. The present cores, which contain small particle size multicomponent SAP particles, or a superabsorbent material A or B, acquire liquids sufficiently fast to avoid problems, like gel blocking, and, therefore, the amount of fluff or pulp in the core can be reduced, or eliminated. A reduction in the amount of the low-density fluff results in a thinner core, and, accordingly, a thinner diaper. Therefore, a core of the present invention can contain at least 0.50% of an SAP, preferably at least 75% of an SAP, and up to 100% of an SAP. In various embodiments, the presence of a fluff or pulp is no longer necessary, or desired.

In addition to a thinner diaper, the present cores also allow an acquisition layer to be omitted from the diaper. The acquisition layer in a diaper typically is a nonwoven or fibrous material, typically having a high degree of void space of “loft,” that assists in the initial absorption of a liquid. The present cores acquire liquid at a sufficient rate such that diapers free of an acquisition layers are practicable.

Surprisingly, tests indicate that, for fluffless and low fluff cores, optimum performance for absorbing different solutions is related to the small particle size range. In particular, the optimum particle size for absorbing JAYCO synthetic urine is a particle size range of about 38 to about 355 μm and a medium particle size of about 200 μm. These small particle size SAP particles would be preferred for use in diaper cores designed for newborn and young infants, e.g., newborns to about one year old.

Similarly, the optimum small particle size SAP determined by the CUP solution method of WO 00/55258, incorporated herein by reference, is a range of about 75 to about 400 μm, and a median particle size of about 240 μm. Such a small particle size SAP would be preferred for older infants and toddlers, e.g., infants about one year old or older.

The following tables summarize results for the above-discussed particle size tests on different test solutions for a fluffless core and core containing fluff.

Fluffless cores containing 9 g LAF (38-300 μm) and compressed at 10,000 pounds for 5 minutes.

| | Insult | | | |
|---|---|---|---|---|
| | Primary | Secondary | Tertiary | Quaternary |
| | 100 mL | 50 mL | 50 mL | 50 mL |
| JAYCO Synthetic Urine | | | | |
| Acquisition Time | 151.63 | 210.81 | 189.08 | 221.11 |
| (sec) | | | | |
| Acquisition Rate | 0.660 | 0.237 | 0.264 | 0.226 |
| (ml/sec) | | | | |
| Rewet (grams | 0.04 | 0.04 | 1.10 | 6.87 |
| CUP Synthetic Urine | | | | |
| Acquisition Time | 201.41 | 238.70 | 532.65 | 780.00 |
| (sec) | | | | |
| Acquisition Rate | 0.496 | 0.209 | 0.094 | 0.064 |
| (ml/sec) | | | | |
| Rewet (grams | 0.09 | 2.55 | 19.28 | 50.00 |
| 0.9% Saline | | | | |
| Acquisition Time | 144.08 | 58.91 | 103.05 | |
| (sec) | | | | |
| Acquisition Rate | 0.694 | 0.849 | 0.485 | |
| (ml/sec) | | | | |
| Rewet (grams | 0.06 | 0.34 | 4.30 | |

Fluff-containing cores containing 9 g ASAP 2300 and 9 g fluff compressed at 10,000 pounds for 5 minutes.

| | Insult | | | |
|---|---|---|---|---|
| | Primary | Secondary | Tertiary | Quaternary |
| | 100 mL | 50 mL | 50 mL | 50 mL |
| JAYCO Synthetic Urine | | | | |
| Acquisition Time | 179.81 | 144.38 | 190.36 | 193.99 |
| (sec) | | | | |
| Acquisition Rate | 0.556 | 0.346 | 0.263 | 0.258 |
| (ml/sec) | | | | |
| Rewet (grams | 0.09 | 1.02 | 9.26 | 20.02 |
| CUP Synthetic Urine | | | | |
| Acquisition Time | 255.75 | 249.50 | 337.25 | 397.25 |
| (sec) | | | | |
| Acquisition Rate | 0.391 | 0.200 | 0.148 | 0.126 |
| (ml/sec) | | | | |
| Rewet (grams | 2.74 | 10.30 | 22.05 | 32.54 |
| 0.9% Saline | | | | |
| Acquisition Time | 304.80 | 290.20 | 350.00 | 423.00 |
| (sec) | | | | |
| Acquisition Rate | 0.328 | 0.172 | 0.143 | 0.118 |
| (ml/sec) | | | | |
| Rewet (grams | 0.02 | 0.38 | 5.78 | 24.20 |

Many modifications and variations of the invention as hereinbefore set forth can be made without departing from the spirit and scope thereof and, therefore, only such limitations should be imposed as are indicated by the appended claims.

1.-28. (Cancelled)

29. A particulate superabsorbent polymer composition comprising
(a) at least one acidic water-absorbing resin neutralized 0% to 50%, and
(b) at least one basic water-absorbing resin neutralized 0% to 50%,
wherein the particles of the particulate superabsorbent polymer have a particle size of about 38 to about 300 μm and a median particle size of less than about 180 μm.

30. The composition of claim 29 comprising discrete particles of the acidic resin and discrete particles of the basic resin.

31. The composition of claim 29 wherein each particle has at least one microdomain of the acidic resin in contact with, or in close proximity to, at least one microdomain of the basic resin.

32. The composition of claim 31 further comprising a matrix resin.

33. The composition of claim 29 comprising about 10% to about 90%, by weight, acidic resin and about 10% to about 90%, by weight, basic resin.

34. The composition of claim 29 wherein the acidic resin and basic resin, independently, are neutralized 0%.

35. The composition of claim 29 wherein the particles have a particle size of about 75 to about 275 μm.

36. The composition of claim 29 wherein the particles have a particle size of about 100 to about 250 μm.

37. The composition of claim 29 wherein the particles have a median particle of less than about 150 μm.

38. The composition of claim 29 wherein the particles have a median particle of less than about 125 μm.

39. The composition of claim 29 wherein the acidic water-absorbing resin is selected from the group consisting of polyacrylic acid, a hydrolyzed starch-acrylonitrile graft copolymer, a starch-acrylic acid graft copolymer, a saponified vinyl acetate-acrylic ester copolymer, a hydrolyzed acrylonitrile polymer, a hydrolyzed acrylamide copolymer, an ethylene-maleic anhydride copolymer, an isobutylene-maleic anhydride copolymer, a poly(vinylphosphonic acid), a poly(vinylsulfonic acid), a poly(vinylphosphoric acid), a poly(vinylsulfuric acid), a sulfonated polystyrene, a poly(aspartic acid), a poly(lactic acid), and mixtures thereof.

40. The composition of claim 29 wherein the basic water-absorbing resin is selected from the group consisting of a poly(vinylamine), a poly(dialkylaminoalkyl(meth)acrylamide), a polymer prepared from the ester analog of an N-(dialkylamino(meth)acrylamide), a polyethylenimine, a poly(vinylguanidine), a poly(allylguanidine), a poly(allylamine), a poly(dimethyldialkylammonium hydroxide), a guanidine-modified polystyrene, a quaternized polystyrene, a quaternized poly(meth)acrylamide or ester analog thereof, poly(vinyl alcohol-co-vinylamine), and mixtures thereof.

41. The composition of claim 29 containing about 50% to 100%, by weight, of basic resin plus acidic resin.

42. The composition of claim 29 wherein the acidic resin and/or basic resin is annealed at a temperature of about 65° C. to about 150° C. for about 20 minutes to about 16 hours.

43. The composition of claim 29 wherein the acidic resin and/or basic resin is surface crosslinked with up to about 1% by weight of the particle of a surface crosslinking agent.

44. The composition of claim 29 wherein the acidic resin contains a plurality of carboxylic acid, sulfonic acid, sulfuric acid, phosphonic acid, or phosphoric acid groups, or a mixture thereof.

45. A method of absorbing an aqueous medium comprising contacting the medium with a composition of claim 29.

46. The method of claim 45 wherein the aqueous medium contains electrolytes.

47. The method of claim 46 wherein the electrolyte-containing aqueous medium is selected from the group consisting of urine, saline, menses, and blood.

48. An absorbent article comprising a composition of claim 29.

49. The article of claim 48 wherein the article is a diaper or a catamenial device.

50. A diaper having a core, said core comprising at least 15% by weight of a particulate superabsorbent polymer composition comprising
(a) at least one acidic water-absorbing resin neutralized 0% to 50%, and
(b) at least one basic water-absorbing resin neutralized 0% to 50%,
wherein the particles of the particulate superabsorbent polymer have a particle size of about 38 to about 300 μm and a median particle size of less than about 180 μm.

51. The diaper of claim 50 wherein the core comprises at least 50% by weight of the composition.

52. The diaper of claim 50 wherein the core comprises at least 75% by weight of the composition.

53. The diaper of claim 50 wherein the core comprises 100% by weight of the composition.

54. The diaper of claim 50 further comprising a topsheet in contact with a first surface of the core, and a backsheet in contact with a second surface of the core, said second core surface opposite from said first core surface.

55. The diaper of claim 54 further comprising an acquisition layer disposed between the topsheet and the core.

56. The diaper of claim 54 wherein the diaper is free of an acquisition layer.
